(19) United States
(12) Patent Application Publication
(10) Pub. No.: US 2011/0053849 A1
(43) Pub. Date: Mar. 3, 2011

(54) REGULATION OF LITAF MODULATED CYTOKINE PRODUCTION BY A NOVEL P53 SHORT PEPTIDE

(76) Inventors: Salomon Amar, Brookline, MA (US); Xiaoren Tang, Winchester (MA)

(21) Appl. No.: 12/377,545

(22) PCT Filed: Aug. 9, 2007
(86) PCT No.: PCT/US07/17687

§ 371 Date: Nov. 15, 2010

Related U.S. Application Data
(60) Provisional application No. 60/838,217, filed on Aug. 17, 2006.

(51) Int. Cl. A61K 38/08; C07K 7/06; C07H 21/04; C12N 15/63; C07K 16/00; C07K 7/08; C07K 14/00; C12N 5/00; C40B 30/04; A61P 3/10; A61P 29/00; A61P 17/02

(52) U.S. Cl. 514/13.3; 530/329; 536/23.1; 435/320.1; 530/387.9; 530/328; 530/327; 530/326; 530/325; 530/324; 530/350; 435/375; 435/455; 506/9

(57) ABSTRACT

The present invention relates to a novel isolated and purified peptide of the sequence KQSQHMT [SEQ ID NO: 1], nucleic acid sequences encoding said peptide sequence and capable of expressing said sequence as an exogenous protein in a target cell; as well as methods for the reduction or inhibition of LITAF activity by transfecting the peptide KQSQHMT [SEQ ID NO: 1] or a nucleic acid capable of expressing said peptide, into a target cell. The present invention also relates to the use of the novel peptide (and corresponding nucleotide sequences) of the present invention for the regulation of cytokine expression in target cells. The present invention also relates to the use of the novel peptide (and corresponding nucleotide sequences) of the present invention for the regulation of inflammatory responses in mammals.

### GOVERNMENT SUPPORT

This invention was made with Government Support under Contract No. NIDCR DE14079 awarded by the National Institute of Health to Salomon Amar. The Government has certain rights in the invention.

### FIELD OF THE INVENTION

This invention relates to a mutant p53 short peptide useful, for example, in the regulation of the production of cytokines and in the regulation of LPS-induced LITAF and TNF-alpha production.

### BACKGROUND

Cytokines are critical players in both the innate and adaptive immune systems. They activate complement, trigger phagocytosis, raise body temperature and activate B and T cells. All of these functions are vital to host defense but cytokine expression must be carefully regulated because these mediators have both favorable and deleterious effects. For example, overproduction of cytokines is thought to contribute to the development of diseases such as diabetes, atherosclerosis and rheumatoid arthritis as well as contribute to inappropriate allergic reactions (Eu, et al., Biochemistry, 39:1375-1384, 2000; Rapoport, et al., Cytokine, 30:219-227, 2005). Conversely, insufficient production of cytokines, resulting in immune suppression, can allow mild opportunistic infections to progress and even to become deadly (Bronke, et al., Immunol Lett, 97:215-224; Cecere, et al., Panminerva Med, 46:171-187, 2004; Nicolle, Lab Invest, 84:1305-1321, 2004). Therefore, understanding the control of cytokine gene expression is crucial to understanding many disease states.

The p53 protein helps to maintain the genomic stability of cells thereby reducing the probability that a cancer will develop. Loss of p53 function is frequently associated with a broad spectrum of human cancers (Nemoto, S, et al., Nutrient availability regulates SIRT1 through a fork head-dependent pathway, Science, 306:2105-2108, 2004). The p53 protein functions as a sequence-specific DNA-binding factor that can activate genes whose promoters contain a p53 response element (Kem, S. E., et al., Identification of p53 as a sequence-specific DNA-binding protein, Science, 252:1708-1711, 1991; Bode, A. M. and Z. Dong, Post-translational modification of p53 in tumorigenesis, Nat. Rev. Cancer, 4:793-805, 2004; Lee, K. C., et al., p53-Mediated Repression of Alpha-Fetoprotein Gene Expression by Specific DNA Binding, Mol. Cell. Biol., 19:1279-1288, 1999; Yin, Y., et al., Human RAD9 checkpoint control/proapoptotic protein can activate transcription of p21, Proc. Natl. Acad. Sci. USA, 101:8864-8869, 2004; Gu, L., et al., MDM2 induces NF-kB/p65 expression transcriptionally through Sp1-binding sites: a novel, p53-independent role of MDM2 in doxorubicin resistance in acute lymphoblastic leukemia, Blood, 99:3367-3375, 2002).

In U.S. Pat. No. 6,566,501 (to Amar, et al., which is herein incorporated by reference) the protein LITAF was described. LITAF is a transcription factor that is instrumental in the regulation of certain cytokines, either directly or indirectly, especially TNF-alpha, IL-1-beta and VEGF. Inhibition of LITAF mRNA expression resulted in a reduction of TNF-alpha and IL-1-beta expression and an increase in VEGF expression. The binding site for LITAF has been found, for example, within the TNF-alpha promoter region. The regulation of LITAF has also been shown to be critical for the regulation of angiogenesis (provisional application Ser. No. 60/655,851 which is incorporated herein by reference)

Regulation of inflammatory cytokines and the role that p53 may play in this regulation is poorly understood. Therefore, what is needed are new compositions and methods of i) deducing the involvement of p53 in the regulation of inflammatory cytokines and ii) modulating the activation of inflammatory cytokines.

### SUMMARY OF THE INVENTION

The present invention relates to a novel peptide of the septamer sequence KQSQHMT [SEQ ID NO: 1] as well as protein sequences comprising the novel peptide sequence KQSQHMT [SEQ ID NO: 1]. This sequence comprises amino acids 164-170 of the p53 protein. This peptide sequence [SEQ ID NO: 1], and peptides comprising SEQ ID NO: 1, have been identified herein as a regulator of LITAF promoter activity. Thus, the sequence KQSQHMT [SEQ ID NO: 1] can be used to modulate the function of the LITAF gene as well as those activates dependent upon the activation of the LITAF gene. Non-limiting examples of those activities include the modulation of the expression of the cytokines TNF-alpha, IL-1-beta, VEGF, IL-2, IL-5, IL-7, IL-12, Eotaxin, IL-15, IFN-γ, IP-10 (human interferon-inducible protein-10), IL-6, RANTES and IL-1ra.

Furthermore, the present invention relates to the use of the novel peptide sequence KQSQHMT [SEQ ID NO: 1] for the regulation of angiogenesis. In this regard and although the present invention is not limited by theory, it is believed that the inhibition of LITAF by [SEQ ID NO: 1] and peptides comprising [SEQ ID NO: 1] leads to a decrease in the expression of LITAF. This decrease in available LITAF changes the balance between STAT6B and LITAF in the cell. A relative increase of STAT6B compared to LITAF has been shown to result in an increase in the expression of VEGF which, in turn, modulates angiogenesis (see, U.S. Pat. No. 6,566,501 to Amar, et al. and U.S. Patent Provisional Application Ser. No. 60/655,851 which is incorporated herein by reference).

The present invention also relates to nucleic acid sequences that encode the peptide sequence KQSQHMT [SEQ ID NO: 1].

The present invention also relates to the use of the peptides and nucleotide sequences of the present invention, or derivatives thereof, for the regulation of cytokine expression in organisms, tissues, cells, cell-free systems and diagnostic or assay systems and protocols. In this regard, the peptides of the present invention or nucleic acid sequences encoding said peptides of the present invention might be transfected into cells, tissues or organisms for the purpose of modulating cytokine activity. Non-limiting examples of the cytokines to be regulated include, for example, TNF-alpha, IL-1-beta, VEGF, IL-2, IL-5, IL-7, IL-12, Eotaxin, IL-15, IFN-γ, IP-10 (human interferon-inducible protein-10), IL-6, RANTES and IL-1ra.

Also in this regard, the present invention relates to the regulation of angiogenesis by the proteins or protein derivatives of the present invention via the regulation of cytokine expression as well as pharmaceutical compositions comprising the proteins or protein derivatives of the present invention.

### BRIEF DESCRIPTION OF THE DRAWINGS

FIG. 1 shows the nucleotide sequence of the LITAF promoter.

FIG. 2 shows a diagram of the LITAF promoter constructs designed for the analysis of promoter activities.

FIG. 3 shows EMSA of the protein-DNA interaction of p53 and the LITAF promoter.

FIG. 4 (A-C) shows the effects of p53 on LITAF promoter activity in H1299 cells.

FIG. 5 shows (A) a diagram of major p53 constructs and (B) the analysis of their effects on LITAF promoter activity.

FIG. 6 shows the effects of transfected p53 constructs on endogenous LITAF gene expression in p53-null H1299 cells.

FIG. 7 shows a luciferase assay using p53 peptides.

FIGS. 8 (A & B) shows the effects of p53 short peptide (pep12) on LPS-induced endogenous LITAF gene expression in human monocytes.

FIGS. 9A, 9B and 9C show results of a Bioplex® human monocyte assay.

### DESCRIPTION OF THE INVENTION

The present invention relates to a novel p53 short peptide [SEQ ID NO: 1] that is involved in the regulation of LPS-induced TNF production as well as compositions derived from the novel peptide and methods for the use of such compositions in modulating cytokine expression in a cell. The present invention is based in part on the identification of a novel peptide (KQSQHMT [SEQ ID NO: 1]). Although the present invention is not limited to any particular theory, the novel sequence of the present invention (and derivatives of the sequence) is believed to bind to the LITAF gene promoter sequence thereby reducing and/or inhibiting LITAF gene transcription. Additionally, the invention provides the use of SEQ ID NO: 1 for modulating the expression of genes other than LITAF. That is, genes that also have a p53 response element or other SEQ ID NO: 1 interactive nucleotide sequence in or near that gene's promoter region.

The present invention also relates to the introduction of the short p53 peptide of the present invention into a cell or cells. The novel sequence of the present invention [SEQ ID NO: 1], or derivatives thereof, may be introduced into a cell or cells by any of the methods known in the art. Methods of introducing proteins into a cell or cells are well known in the art and are provided in detail in, for example, Sambrook, J., Fritsch, E. F. and Maniatis, T., Molecular Cloning: A Laboratory Manual. Cold Spring Harbor Laboratory Press, NY, Vol. 1, 2, 3 (1989), which is incorporated herein by reference. In brief, proteins may be introduced into cells in two general ways. The first is with the transfection of nucleic acids expressing the desired protein. Once inside the cell the protein is produced by the cell's own transcription and translation components. The second is with the transfection of the actual protein into the cell.

The transfection of nucleic acids is well known in the art. The nucleic acid sequence (for example, the nucleic acid sequences of the present invention) encoding the desired protein is operably inserted into an expression vector suitable for the transcription and translation of the nucleic acid sequence into the desired protein. A large number of expression vectors are commercially available (e.g., pCAT, Promega, Madison, Wis.; pBlueScript and pCMV, Stratagene, La Jolla, Calif.). The splicing of a specific nucleotide sequence into an expression vector is also well know in the art as is referenced above in Sambrook, et al. Suitable transfection methods include DEAE-dextran, calcium phosphate precipitation, Lipofectamine™ (Invitrogen, Carlsbad, Calif.), Profectin™ (Promega, Madison, Wis.) and other liposome methods, direct microinjection, electroporation and bioloastic particle delivery, for example. Any primary cell type or any cell line may be used for the present invention. The short p53 peptide of the present invention may also have additional amino acids added to either of the amino or carboxy terminals. Anywhere form zero to 100 or more amino acids may be added to one or both peptide terminals. Examples of sequences to be added may be, for example, tags for the detection of or for determining the location(s) of the peptide within the cell. Amino acid sequences for tethering the short p53 peptide of the present invention to a specific location in a cell or tissue or to a specific surface (e.g., the surface in a reaction vessel). Non-limiting examples of suitable tethering sequences include the lambda bacteriophage antiterminator protein N (lambdaN-(1-22) or lambdaN peptide) (J. Baron-Benhamou, et al., Methods Mol Biol., 257:135-154, 2004, which is herein incorporated by reference), a glycine-serine tether (U.S. Pat. No. 7,074,557, which is herein incorporated by reference). Or, sequences for aiding in the transport of the peptide through, for example, cell or organelle membranes or for transport through selective pores in organelle or cell membranes. Many tags, tethering sequences and sequences to aid in transport into and through cells or organelles are well known to those practiced in the art.

Transfection of the actual peptides of the present invention (i.e., proteins comprising SEQ ID NO: 1) may take place by, for example, transport (active or passive) or by microinjection. Active transport is a process whereby cells absorb material from the outside the cell by engulfing it with the cell membrane. Passive transport may also take place via the passage of peptide fragments, for example, into the cell through pores. Cells frequently transport particles and, especially, proteins and protein fragments into the cell's cytoplasm. Transport may be specific via, for example, specific receptors or it may be more general. With general forms of active transport the cell engulfs constituents from the extracellular milieu. This is often referred to as pinocytosis. Pinocytosis (literally, cell-drinking) is the invagination of the cell membrane to form a pocket filled with extracellular fluid (and molecules within it). The pocket then pinches off to form a vesicle, and the vesicle ruptures to release its contents into the cytoplasm.

In addition to the techniques given directly above, transfection of the proteins of the present invention into cells may also be executed by the transfection of nucleic acids that express the peptide(s) of the present invention into cells, as given above. Any primary cell type or any cell line may be used as a recipient for the peptides and nucleic acid sequences of the present invention. The techniques of nucleic acid transfection are well known in the art (see, for example, Sambrooke, et al., herein incorporated by reference).

Peptides comprising SEQ ID NO: 1 also have the ability to modulate cytokine expression in the methods of the present invention (see, Examples). A biologically active peptide may further be a protein, polypeptide or peptide. As defined in this invention, the terms “protein,” “peptide” or “polypeptide” are interchangeable and refer to a sequence of two of more amino acids with or without additional modifications such as, but not limited to, glycosylation.

The present invention also relates to methods for the modulation of cytokine expression. These may include, for example, introducing into a cytokine-responsive cell a composition comprising SEQ ID NO: 1. In this method, SEQ ID NO: 1 is to be introduced into the cell in an amount effective to modulate cytokine expression. Amounts may vary depending, for example, the target cell or tissue type but can be determined easily via titration of the peptide or expression construct used. The cytokine to be modulated may be, for example, TNF-α and/or IL-1β. Introduction of SEQ ID NO: 1 into the cell decreases expression of TNF-α and IL-1β and increases expression of VEGF and, as such, this method may be used to effect any of the cellular processes resulting from the same.

One of skill in the art will recognize that a biologically active peptide comprising KQSQHMT [SEQ ID NO: 1] may be used in lieu of the septamer peptide of the present invention. A “biologically active peptide” is intended to encompass any mimetic, truncation, deletion and/or substitution or elongation of the peptide sequence of the present invention. In one embodiment, the peptide of SEQ ID NO: 1 comprises an additional 0-100 amino acids in sequence with one or both of the amino- or carboxy-terminals of the peptide.

One of skill in the art will recognize that the peptide KQSQHMT or proteins comprising the active peptide KQSQHMT may be introduced into a cell by various means in the methods of the present invention. A cell may be contacted directly with the peptide KQSQHMT or proteins comprising active KQSQHMT under conditions for cellular uptake. Such conditions include but are not limited to injection and calcium chloride mediated uptake, electroporation, microinjection, etc. Alternatively, a target cell (e.g., a cytokine-responsive cell) may express exogenous KQSQHMT or proteins comprising active KQSQHMT from an introduced exogenous construct harboring an expressible cDNA construct or constructs, as discussed above.

In the methods of the present invention, a composition comprising KQSQHMT or proteins comprising KQSQHMT may be administered to an animal or individual in a physiologically acceptable carrier in a therapeutically effective amount. Said compound or compounds may be administered alone or in combination with other therapies and may be delivered intravenously, subcutaneously or orally to an animal. Administration may be systemic although local administration may be preferable.

It is an object of the present invention to employ the methods disclosed herein for modulating cellular responses to cytokine expression. The methods of the present invention may be used to study and/or treat diseases associated with aberrant cytokine signaling. It is known in the art that cytokine signaling is involved in pro-inflammatory and anti-inflammatory responses to pathogens and in cellular proliferation and differentiation in a variety of cells. Thus, methods disclosed herein for modulating cytokine signaling may be used to alter these and other cytokine-dependent processes in normal and/or abnormal cells.

TNF-alpha (tumor necrosis factor-alpha) is a cytokine released by, for example, white blood cells in the course of damage by, for example, infection. Unregulated release of TNF-alpha has been implemented in the etiology of several diseases including, for example, various autoimmune disorders such as rheumatoid arthritis, Crohn's disease and psoriasis.

Interleukin-1-beta, (IL-1-beta) is a cytokine implemented in numerous diseases including acute phase response (the near immediate response by the immune system in injury that is typically characterized by, e.g., redness and swelling. It is secreted by is secreted by macrophages, monocytes and dendritic cells. The secretion of both TNF-alpha and IL-1-beta are regulated by LITAF expression. Thus, the inhibition of LITAF expression by, for example, SEQ ID NO: 1 can be used to modulate the secretion of both these cytokines since decreases in LITAF expression leads to decreases in both TNF-alpha and Il-1-beta expression.

Thus, the modulation of cytokine signaling via the compositions and methods of the present invention may also be used to modulate the immune response of an animal or individual to an antigen, or to treat diseases or repair of damage caused by such as diabetes or inflammatory diseases.

Also provided herein are compositions and methods for the treatment of non-healing ulcers, for example, in the context of diabetes. Diabetes typically may cause such ulcers (e.g., tissue damage of the kidney) or be the cause for wounding (e.g., amputation of limbs as a result of disease progression). The compositions and methods of the present invention are ideally suited for the treatment of such conditions via the promotion of angiogenesis.

The present invention relates to the interaction of LITAF with a short peptide of the oncogene product p53. Human p53 is a 393-amino acid nuclear transcription factor. Although the present invention is not limited to any particular theory, it is believed that after binding specifically to the promoter regions of its target genes, p53 activates or inhibits their expression. It is shown in the present invention that p53 directly binds the LITAF promoter region. This binding significantly inhibits LITAF promoter activity and, therefore, suppresses LITAF expression. Therefore, one aspect of the present invention relates to the screening of molecules that may inhibit (or increase) the binding of the p53 short peptide of the present invention to the LITAF promoter. For example, in one embodiment, molecules from a library of small molecules are used to form a reaction mixture comprising a small molecule and p53 (or an active fragment thereof, i.e., SEQ ID NO: 1) and a DNA sequence comprising the LITAF promoter region. The mixture is incubated for a length of time and under conditions appropriate for the binding of p53 to the LITAF promoter region. Next, the binding of p53 to the LITAF promoter region is determined (e.g., by electrophoresis or Western blotting). The amount of binding is compared to an identical reaction mixture wherein the small molecule suspected of inhibiting p53 binding to the LITAF promoter region was not added, wherein the decrease in binding in the reaction mixture is indicative of the inhibition of the binding of p53 to the LITAF promoter region.

The present invention also relates to compounds and methods for the stimulation of angiogenesis by administering to an organism a suitable amount of the short p53 peptide of the present invention (or an active derivative thereof). Said peptide (or active derivative) may be administered either locally or systemically and may be administered as liquids, pills injections, etc. Suitable excipients may also be added to the identified compound(s) for ease of manufacture, stability, ease of administration, etc. Likewise, in one embodiment, the present invention contemplates the stimulation of angiogenesis by the administration to an organism of a compound capable of inhibiting the transcription of LITAF to the extent necessary to create a measurable stimulation of angiogenesis.

While specific embodiments of the present invention have been described, it will be apparent to those skilled in the art that various modifications thereto can be made without departing from the spirit and scope of the invention as defined in the examples and appended claims.

### EXAMPLES

### Materials and Methods

Cell Culture. All bacterial cloning constructs used E. coli strain DH5α (Invitrogen). U₂OS human osteosarcoma cells (−53 wild-type; ATCC number: HTB-96; ATCC, Bethesda, Md.) were grown in DMEM with 10% FBS. Human H1299, a p53-null non-small cell lung cancer cell line (ATCC number: CRL-5803), which contains a homozygous deletion of the p53 gene, was maintained in RPMI 1640 medium supplemented with 2 mM L-glutamine and adjusted to contain 10 mM HEPES, 1 mM sodium pyruvate, 4.5 g/L glucose, 1.5 g/L sodium bicarbonate and 10% FBS. Human monocytes, purchased from AB Inc., (Columbia, Md.) were grow in RPMI 1640 medium supplemented with 10% FBS. All human cell cultures were maintained inn 37° C. humidified atmosphere containing 5% CO₂.

Plasmid constructs. (I) The clone, p53-1, which contains a full length human p53 gene and expresses wild-type p53 protein in mammalian cells, was kindly provided by Dr. Belt Vogelstein (The Johns Hopkins Oncology Center, Baltimore, Md.). A series of human p53 deletions were constructed and the major clones named A12, B1, C6, D10 or p53LFB12, as follows: (a) A12 contained the coordinating amino acids (aa) of p53 (1-393Δ101-169); the first mutant DNA fragment was generated by PCR with the primer pair 5′-ATGGAGGAGCCGCAGTCAGAT-3′ [SEQ ID NO: 2] and 5′-CCGTCATGTGCTGGGAAGGGACAGAAGATG-3′ [SEQ ID NO: 3]. The second DNA fragment was generated by PCR with the primer pair 5′-CACATGACGGAGGTTGTGAG-3′ [SEQ ID NO: 3] and 5′-TCAGTCTGAGTCAGGCCCT-3′ [SEQ ID NO: 4]. Both the first and second PCR-generated. DNA fragments were purified and diluted as template to 1 ng per reaction, and amplified by PCR with the primer pair 5′-ATGGAGGAGCCGCAGTCAGAT-3′ [SEQ ID NO: 5] and 5′-TCAGTCTGAGTCAGGCCCT-3′ [SEQ ID NO: 6]. Finally, the p53 site mutant DNA fragment was inserted into the pcDNA3.1/V5-His TOPO vector. (b) B1 contained coordinating aa of p53 (1-393Δ167-234); the first mutant DNA fragment was generated by PCR with the primer pair 5′-ATGGAGGAGCCGCAGTCAGAT-3′ [SEQ. ID NO: 7] and 5′-ACATGTAGTTCTGTGACTGCTTGTAGATGG-3′ [SEQ ID NO: 8]. The second DNA fragment was generated by PCR with the primer pair 5′-AACTACATGTGTAACAGTTCC-3′ [SEQ ID NO: 9] and 5′-TCAGTCTGAGTCAGGCCCT-3′ [SEQ ID NO: 10]. Both the first and second PCR-generated DNA fragments were purified and diluted as template to 1 ng per reaction, and amplified by PCR with the primer pair 5′-ATGGAGGAGCCGCAGTCAGAT-3′ [SEQ ID NO: 11] and 5′-TCAGTCTGAGTCAGGCCCT-3′ [SEQ ID NO: 12]. Finally, the p53 site mutant DNA fragment was inserted into the pcDNA3.1/V5-His TOPO vector. (c) C6 contained coordinating aa of p53 (1-393Δ235-300); the first mutant DNA fragment was generated by PCR with the primer pair 5′-ATGGAGGAGCCGCAGTCAGAT-3′ [SEQ ID NO: 13] and 5′-TGCTCCCTGGGTAGTGGATGGTGGTACAGT-3′ [SEQ ID NO: 14]. The second DNA fragment was generated by PCR with the primer pair 5′-CCAGGGAGCACTAAGCGAGCA-3′ [SEQ ID NO: 15] and 5′-TCAGTCTGAGTCAGGCCCT-3′ [SEQ ID NO: 16]. Both the first and second PCR-generated DNA fragments were purified and diluted as template to 1 ng per reaction, and amplified by PCR with the primer pair 5′-ATGGAGGAGCCGCAGTCAGAT-3′ [SEQ ID NO: 17] and 5′-TCAGTCTGAGTCAGGCCCT-3′ [SEQ ID NO: 18]. Finally, the p53 site mutant DNA fragment was inserted into the pcDNA3.1/V5-His TOPO vector. (d) D10 contained coordinating aa of p53 (1-393Δ156-178); the first mutant DNA fragment was generated by PCR with the primer pair 5′-ATGGAGGAGCCGCAGTCAGAT-3′ [SEQ ID NO: 19] and 5′-GGTGCCGGGCGGGGGTGTGGA-3′ [SEQ ID NO: 20]. The second DNA fragment was generated by PCR with the primer pair 5′-CATGAGCGCTGCTCA-3′ [SEQ ID NO: 21] and 5′-TCAGTCTGAGTCAGGCCCT-3′ [SEQ ID NO: 22]. Both the first and second PCR-generated DNA fragments were purified and diluted as template to 1 ng per reaction, and amplified by PCR with the primer pair 5′-ATGGAGGAGCCGCAGTCAGAT-3′ [SEQ ID NO: 23] and 5′-TCAGTCTGAGTCAGGCCCT-3′ [SEQ ID NO: 24]. Finally, the p53 site mutant DNA fragment was inserted into the pcDNA3.1/V5-His TOPO vector. (e) p53LFB12 contained coordinating aa of p53 from 164 to 170 but added one methionine (M) for initiation; the DNA fragment with the primer pair 5′-ATGAAGCAGTCACAGCACATGACGA-3′ [SEQ ID NO: 25] and 5′-CGTCATGTGCTGTGACTGCTTCATA-3′ [SEQ ID NO: 26] was annealed by heating for 2 minutes at 65° C., then cooling slowly to <35° C. over 15-30 minutes. The annealed DNA fragment was inserted into the pcDNA3.1/V5-His TOPO vector. (f) 12REV contained the coordinating reverse aa of p53LFB12 above from 170 to 164 as a control, but added one methionine (M) for initiation; the DNA fragment with the primer pair 5′-ATGGCAGTACACGACACTGACGAAA-3′ [SEQ ID NO: 27] and 5′-TTCGTCAGTGTCGTGTACTGCCATA-3′ [SEQ ID NO: 28] was annealed by heating for 2 minutes at 65° C., then cooling slowly to <35° C. over 15-30 minutes. The annealed DNA fragment was directly inserted into the pcDNA3.1/V5-His TOPO vector.

(II) Human genomic DNA from THP-1 cells was prepared as described (Yoo, Y. C., et al., Apoptosis in human leukemic cells induced by lactoferricin, a bovine milk protein-derived peptide: involvement of reactive oxygen species, Biochem. Biophys. Res. Commun., 237:624-628, 1997). Subsequently, the full length LITAF promoter DNA was generated from human genomic DNA by PCR with the primer pairs: 5′-CCAGAGGGCCGGGAGCGCCCCA-3′ [SEQ ID NO: 29] and 5′-TTTACCCAGCACCGGCGGTGGA-3′ [SEQ ID NO: 30], and was subcloned into a vector, pGL3-basic (Promega). This recombinant vector, containing a full length LITAF 5′ UTR, was named pGLP990. Deletions of LITAF promoter DNA were generated by PCR using pGLP990 as the template, using the following primer pairs: (a) 5′-CGGGGAACCGGCGATGGTCTC-3′ and 5′-TTTACCCAGCACCGGCGGTGGA-3′ [SEQ ID NO: 31] for the region −700˜+100 of the LITAF promoter; (b) 5′-GCCCCCGCCCCCGTCCCCGCC-3′ [SEQ ID NO: 32] and 5′-TTTACCCAGCACCGGCGGTGGA-3′ [SEQ ID NO: 33] for the region −600˜+100 of the LITAF promoter; (c) 5′-GGCCAGCTCAGACCTCCCGGC-3′[SEQ ID NO: 34] and 5′-TTTACCCAGCACCGGCGGTGGA-3 [SEQ ID NO: 35] for −550˜+100′; (d) 5′-CGGCGCGGGGACGCCGGGGCG-3′ [SEQ ID NO: 36] and 5′-TTTACCCAGCACCGGCGGTGGA-3′ [SEQ ID NO: 37] for −500˜+100; (e) 5′-GGTGGQGCCAGCACCTGCTGG-3′ [SEQ ID NO: 38] and 5′-TTTACCCAGCACCGGCGGTGGA-3′ [SEQ ID NO: 39] for −450˜+100; (f) 5′-GCCTCCTGGGATGCCAGGGGG-3′ [SEQ ID NO: 40] and 5′-TTTACCCAGCACCGGCGGTGGA-3′ [SEQ ID NO: 41] for −400˜+100. These deletion DNA fragments were subcloned into the pGL3-basic vector and named pGLP700, pGLP600, pGLP550, pGLP500, pGLP450 or pGLP400, respectively.

DNA probes. DNA without mutation was amplified from pGLP990 DNA as the template by PCR using the primer pairs 5′-GCCCCCGCCCCCGTCCCCGCC-3′ [SEQ ID NO: 42] and 5′-CCCCAGCCAAGGGCTCAGTGC-3′ [SEQ ID NO: 43] for the region −600˜−400 of LITAF promoter; the primer pairs 5′-GCCCCCGCCCCCGTCCCCGCC-3′ [SEQ ID NO: 44] and 5′-CTGGCGCCACCGGCCCCCCGC-3′ [SEQ ID NO: 45] were used for −600˜−440; and the primer pairs 5′-GCGCCCGAGAGGCCAGCTCAGA-3′[SEQ ID NO: 46] and 5′-CCCCAGCCAAGGGCTCAGTGC-3′ [SEQ ID NO: 47] were used for −560˜−400. DNA containing deletions was generated as follows. The double-stranded oligonucleotide (ds-oligo) was annealed by heating for 2 minutes at 65.C, then cooled slowly to <35.C over 15-30 minutes with the follow primer pairs: 1) 5′-GCCCCCGCCCCCGTCCCCGCCGCCCGGCCCTTTTCTCGGGGCGCCCGAGAGGC CAGCTCAGACCTCCCGGCTCGACAGGCGGCGCGGGCGGCGGTGAGTG-3′ [SEQ ID NO: 48] and 5′-CCCCAGCCAAGGGCTCAGTGCCCGGGGCCCCCAGCAGGTCCCCGGCGGTCCCC GCGCCGCACTCACCGCCGCCCGCGCC-3′ [SEQ ID NO: 49] for −600˜−400Δ−480 ˜−400; 2) 5′-GCCCCCGCCCCCGTCCCCGCCGCCCGGCCCTTTTCTCGGGGCGCCCGAGA GGCCAGCTCAGACCTCCCGGCTCGACAGGCCGGGGACCAG-3′ [SEQ ID NO: 50] and 5′-CCCCAGCCAAGGGCTCAGTGCCCGGGGCCCCCAGCAGGTGCTGGCGCCACCG GCCCCCCGCTGTCTCCCGCTGGTCCCCGGCCTGTCGAG-3′ [SEQ ID NO: 51] for −600˜−400Δ−520˜−480; 3) 5′-GCCCCCGCCCCCGTCCCCGCCGCCCGGCCCTTTTCTCGGGGCGCGGGCGGCGG TGAGTGCGGCGCGGGGACGCCGGGGCGCGGGGACCAG-3′ [SEQ ID NO: 52] and 5′-CCCAGCCAAGGGCTCAGTGCCCGGGGCCCCCAGCAGGTGCTGGCGCCACCGG CCCCCCGCTGTCTCCCGCTGGTCCCCGCGCCCCGGCG-3′ [SEQ ID NO: 53] for −600˜−400Δ−560˜−520. After annealing, each 0.01 μg annealed double strand (ds)-oligo was amplified without additional primers or template by PCR. Finally, the DNAs were purified using a gel extraction kit, (Qiagen) and each DNA species was labeled with [γ³²P]ATP using T4 polynucleotide kinase (Promega) following the manufacturer's instructions. Labeled double-stranded DNA oligos were purified using 0-25 Sephadex columns (Boehringer) and precipitated with ethanol. After centrifugation, the DNA pellets were suspended in 10 μl water and aliquots of each oligo were measured for cpm/μl and used as probes for EMSA, as described below.

Peptides. Synthetic peptides were supplied by Biosynthesis Inc. (Lewisville, Tex.). Pep12 consisted of the p53 sequence KQSQHMT [SEQ ID NO: 1] located in the region from aa 164-170; pepSC served as a negative control peptide and consisted of the randomly scrambled sequence SKMQQTH [SEQ ID NO: 54] (Medusa Random Sample Generator Software, Randombots.com). Both peptides were solubilized in DMSO and delivered into p53-null H1299 or human monocytes by Chariot kit (Active Motif, Carlsbad, Calif.). They were detected by reporter assays and Western blots following the manufacturer's instruction.

Electrophoresis mobility shift assay (EMSA). U₂OS cells were used for EMSA due to its high DNA transfection efficiency. The cell cultures (5×10⁶) were transfected with 1 μg of p53-1 DNA using Lipofectamine 2000 (Invitrogen) for 3 hrs, washed with PBS, then incubated overnight in medium (DMEM+10% FBS) at 37° C., in a humidified atmosphere containing 5% CO₂. The proteins from cells were extracted with lysis buffer (Promega) plus a cocktail of protein inhibitors (Sigma) following the manufacturer's instruction. The proteins were used for EMSA. A reaction mixture contained 10 μg of extract proteins, 1×10⁵ cpm/μl radiolabeled double-stranded oligo DNA probe, 3 μg of poly dI•dC (deoxyinosinic acid•deoxycytidylic acid; Sigma), 5 μg of BSA, 4 μl of gel shift binding 5× buffer (Promega), and nuclease-free water to achieve a final volume of 20 μl. Mixtures were incubated at room temperature for 30 min, followed by electrophoresis on non-denaturing 6% polyacrylamide gels in Tris-borate-EDTA buffer (90 mM Tris-borate/2 mM EDTA HEPES, pH 8.0).

Western blot analysis. Cultures of p53-null human H1299 non-small cell lung cancer cells (5×10⁶) or p53 wildtype human monocytes (1×10⁶) were transfected with DNAs using Lipofectamine 2000 (Invitrogen) for 3 hrs, washed with PBS and further transfected with synthetic p53 peptides using a Chariot kit (Active Motif, Carlsbad, Calif.) as required. Cells were then incubated overnight in RPMI 1640 medium with 10% FBS at 37° C., in a humidified atmosphere containing 5% CO₂. The proteins from the treated cells were extracted with lysis buffer (Promega) plus a cocktail of protein inhibitors (Sigma) following the manufacturer's instructions and suspended in SDS sample buffer, heated at 95° C. for 5 min, then applied to SDS-polyacrylamide gels and detected by Western blotting. Antibodies were purchased from the following vendors: LITAF (611615, BD Biosciences), actin (C-11, Santa Cruz), p53 (FL-393, Santa Cruz) or luciferase (NB 600-307, Novus Bio. Inc).

Luciferase assay. Cultures of U₂OS (5×10⁶), p53-null human H1299 non-small cell lung cancer cells (5×10⁶) or p53 wildtype human monocytes (1×10⁶) were co-transfected with DNAs using Lipofectamine 2000 (Invitrogen) for 3 hrs, washed with PBS, then further treated with peptides by Chariot kit (Active Motif, Carlsbad, Calif.) as required. Cells were then incubated overnight in appropriate media (DMEM or RPMI 1640)+10% FBS at 37° C., in a humidified atmosphere containing 5% CO₂. The β-galactosidase gene was included in all transfections. Cells were harvested and the luciferase activity of each lysate was measured (Turner Designs luminometer model TD-20/20) using a commercial kit (luciferase reporter assay system, Promega) according to the protocol provided by the manufacturer. Finally, the data were normalized to β-galactosidase expression.

ELISA. Human monocytes (1×10⁶) were stimulated with 0.1 μg/ml E. coli LPS for 3 hrs, washed with PBS, then further treated with peptides by Chariot kit (Active Motif, Carlsbad, Calif.). Cells were then incubated overnight in RPMI 1640 medium supplemented with 10% FBS at 37° C., in a humidified atmosphere containing 5% CO₂. Culture supernatants were harvested and centrifuged at 1,500×g to remove cell debris, then concentrations of human TNF-α in the supernatant of each treated or untreated control cell condition were measured by ELISA (Abraxis, Warminster, Pa.). The ELISA immunoreactivity was quantified using a VerSaDoc Imaging System (BIO-RAD) and graphed.

### Example 1

### Investigation of p53/LITAF/TNF Pathway: The Role of p53

It was observed that LITAF is linked to the well known sensor and mediator of damage-induced apoptosis, p53 (Polyak, et al., Nature, 389:300-305, 1997, which is herein incorporated by reference). Human p53 is a 393-amino acid nuclear transcription factor. After binding specifically to the promoter regions of its target genes, p53 activates their expression. This example determined the nature of the signaling involved in p53 accumulation and LITAF mRNA induction after LPS stimulation. It was demonstrated that a direct binding between p53 and LITAF promoter took place. The region on the LITAF promoter where p53 binds was also identified. The specific site required for p53 binding is located between −600 to −480 bp on the LITAF promoter and binding of p53 protein significantly inhibits LITAF promoter activity. These findings suggest that LITAF expression is repressed by.

### Example 2

### Identification of LITAF Promoter Constructs

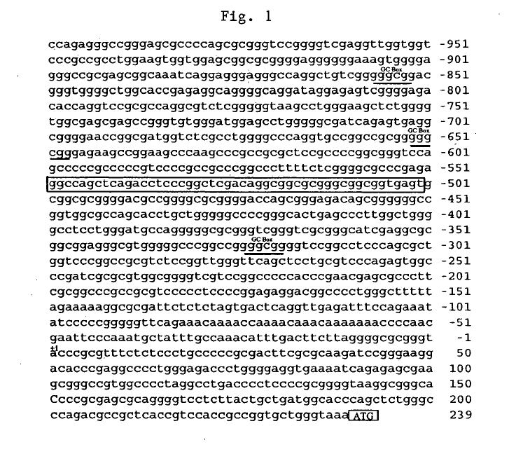

The full-length LITAF promoter DNA (gPLP990) and its derived deletions were subcloned into pGL3-basic, a plasmid lacking a promoter sequence. Sequence analysis of these clones showed that they were almost identical to GenBank accession number AB074031, but there were a few differences: one base was shifted from g to a in position −899, as well as one base “t” was added I n−14; also an additional sequence with 26 bases “aaacaaaaccaaaacaaacaaaaaaa” [SEQ ID NO: 55] was present in the region (−18˜−58) of the human LITAF promoter DNA, based on our sequence data. Finally, the sequence lacking a TATA box but including the presence of multiple GC boxes was indicated (FIG. 1). In FIG. 1, the transcription start site is indicated by a +1 as described (Myokai, F., et al., A novel lipopolysaccharide-induced transcription factor regulating tumor necrosis factor alpha gene expression: molecular cloning, sequencing, characterization and chromosomal assignment, Proc. Natl. Acad. Sci. USA, 96:4518-4523, 1999). The first amino acid of the ORF is indicated by a rectangle. Underlines represent GC bases. The punitive site for p53 binding from −550 to −500 is surrounded with a box.

### Example 3

### Functional Analysis of the LITAF Promoter by 5′ Upstream Deletions

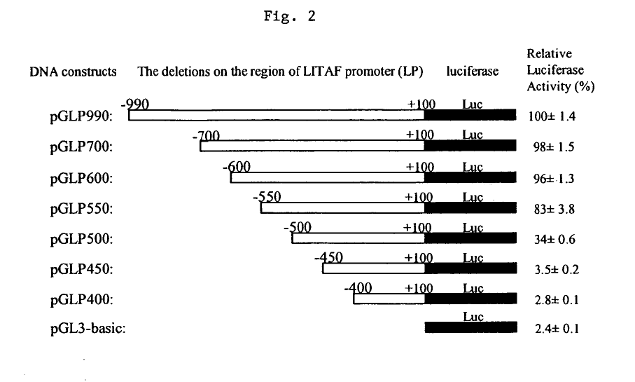

To examine whether the LITAF promoter contains self-transactivation activity, and to further determine which region might respond to this activity, deletion promoter constructs were generated containing the 1,090 bp promoter vector (−990 to +100) and a series of 5′ upstream sequence deletions. Each of these constructs, the full-length luciferase reporter construct pGLP990 and deletion constructs pGLP700, pGLP600, pGLP550, pGLP500, pGLP450 and pGLP400 (FIG. 2), were individually transfected into U₂OS cells. To make the constructs, different lengths of the LITAF promoter DNA were fused to a promoter-less luciferase reporter vector, pGL3-basic. The open box represents the LITAF promoter and the filled box represents the luciferase reported gene (Luc). The full-length construct containing a sequence from the region −990 bp to +100 bp of LITAF promoter yielded the highest luciferase activity, which was set at 100% for comparison with deletion constructs. Triplicate assays were performed. Values were normalized by β-gal assay.

The protein extract from the pGLP990-transfected cells was assigned the maximal promoter activity value of 100%. The relative activity that could be detected decreased gradually: pGLP700 (98%), pGLP600 (96%), pGLP550 (83%), pGLP500 (34%), pGLP450 (3.5%) and pGLP400 (2.8%). Thus, the results showed that the element required for self-transactivation activity was mainly located in the region upstream to −500 as indicated by the open bars in FIG. 2.

### Example 4

### p53 Binding Activity

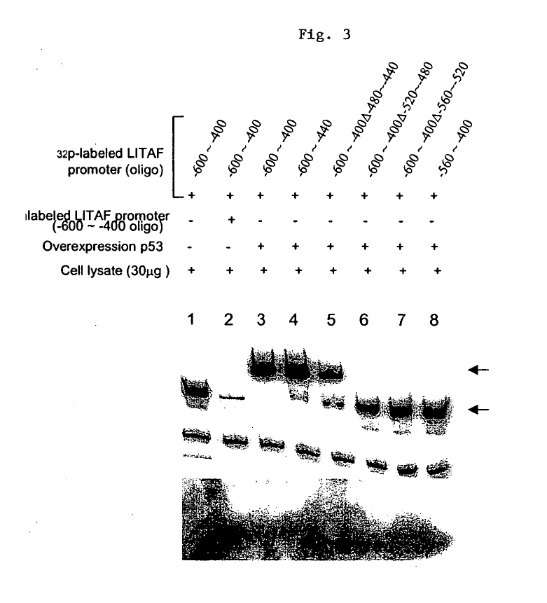

To analyze binding of p53 to the LITAF promoter sequence, EMSA was performed with the protein extract from p53-1-transfected (p53-1 is wild type p53) U₂SO cells (a human bone osteosarcoma epithelial cell line), (FIG. 3, lanes 3-8) in the appropriate buffer with one DNA probe (−600˜−400, −600˜−440, −550˜−400, −600˜−400Δ−440˜−440, −600˜−400Δ−520˜−480 or −600˜−400Δ−560˜−520; see, infra) in separate lanes. Lane 2 also contained a 50-fold excess of unlabeled competitor. The DNA amplified from a different region of the LITAF promoter was labeled with [³²P]ATP (Tang, et al., Identification and functional characterization of a novel binding site on TNF-alpha promoter, Proc. Natl. Acad. Sci USA, 100:4096-4101, 2003) and used as a probe (1×10⁵ cmp/μl) in lanes 1-3 (−600˜−400), lane 5 (−600˜−400Δ−480˜−440), lane 6 (−600˜−400Δ−520˜−480), lane 7 (−600˜−400Δ−560˜−520) and lane 8 (−560˜−400). Protein (10 μg) extracted from untransfected U₂OS cells (lacking p53) (lanes (1-2) or p53-transfected U₂OS cells (lanes 3-8) was added to the appropriate reaction buffer with the DNA probe above in each lane. In lane 2, a 50-fold excess of unlabeled competitor was also added. The shifted DNA bands are indicated by arrows.

Clearly, the deletion of −480-−440 (lane 5) did not significantly affect the DNA binding. However, the deletions ranging from −520-−480 (lane 6), −550-−520 (lane 7) or −600-−560 (lane 8) completely blocked the DNA binding. The results suggest the p53 binds within the region from −600-−480 of the LITAF promoter.

### Example 5

### p53-Dependent Inhibition of LITAF Promoter Activity

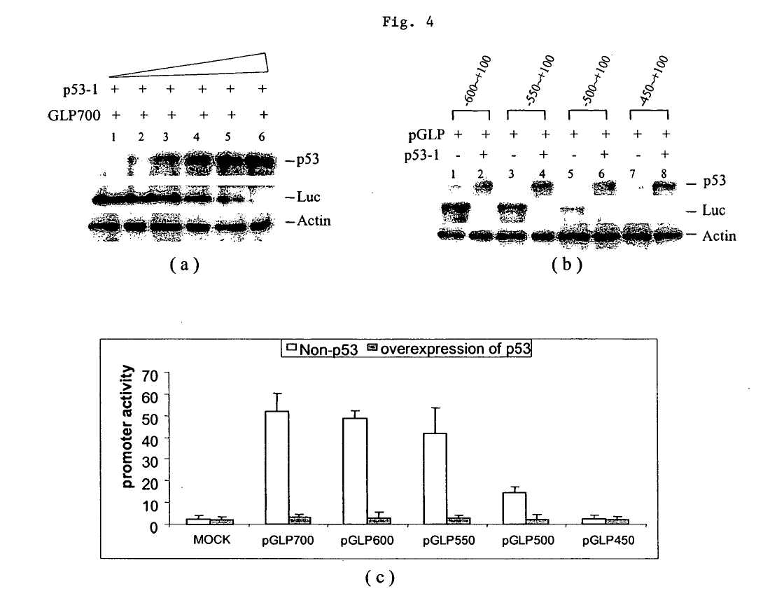

To examine whether LITAF promoter activity could be regulated by p53, p53-null H1299 cells (p53^{-/-}) were co-transfected with pGLP700 and p53-1 and then protein extracts were analyzed by Western blot. In the dose response assay of FIG. 4A, cells were transiently transfected overnight with 0.5 μg of pGLP700 (each lane), plus 0 μg (lane 1), 0.1 μg (lane 2), 0.2 μg (lane 3), 0.3 μg (lane 4), 0.4 μg (lane 5) or 0.5 μg (lane 6) of p53-1 DNA and quantities of pcDNA3 plasmid DNA to equalize the total amount of transected DNA for each condition (1 μg). Cells were incubated overnight at 37° C. Extracts were analyzed by Western blot for the presence of p53, luciferase or actin. Triplicate tests were performed. FIG. 4A is representative of the three tests.

The results (FIG. 4A) clearly show that the transient transfection of different concentrations of p53-1: 0.1 μg (lane 2); 0.2 μg (lane 3); 0.3 μg (lane 4); 0.4 μg (lane 5) and 0.5 μg (lane 6) caused a gradual decrease of the LITAF promoter-activated luciferase gene expression.

To further characterize the effect of p53 on the responsive region of the LITAF promoter, p53-null H1299 cells were co-transfected with 0.5 μg of pGLP700, pGLKP600, pGLP550, pGLP500, pGLP450 or pGLP400, plus 0.5 μg of p53-1. Western blot analysis was performed on extracted aliquots (from the cells described in paragraphs [0077] and [0078]) containing pGLP600 (lanes 1 and 2), pGLP550 (lanes 3 and 4), pGLP500 (lanes 5 and 6) or pGLP450 (lanes 7 and 8), plus 0.5 μg of p53-1 (lanes 2, 4, 6 and 8) or 1 μg pcDNA3 (lanes 1, 3, 5 and 7). A total of 60 μg of protein extracts was loaded per lane. Blots were probed with the following antibodies: p53 (FL-393, Santa Cruz), luciferase (NB 600-307, Novus Bio, Inc.) or actin (C-11, Santa Cruz) as control.

As shown in FIG. 4B (lanes 2, 4 or 6), luciferase gene expression induced by LITAF promoter in the responsive region from −550 to −500 was completely blocked due to transfection of 0.5 μg of p53-1. The result of directly measuring luciferase production also showed the inhibition of gene expression when functional p53 was present (FIG. 4C). In the assays shown in FIG. 4C, luciferase expression was measured in H1299 cell lacking p53 that had been co-transfected with 1 μg DNA including luciferase reporter constructs plus p53-1. The cells cultured for 16 hours after DNA transfection were harvested and their proteins were purified. The concentration of luciferase from each test was individually measured by a luciferase reporter system (Promega, Madison, Wis.). Triplicate assays were performed. Values were normalized to β-gal production. The figure compares effects of p53 on promoter activity in cells containing p53 (filled holes) or lacking p53 (open box).

### Example 6

### p53 Binding Domain

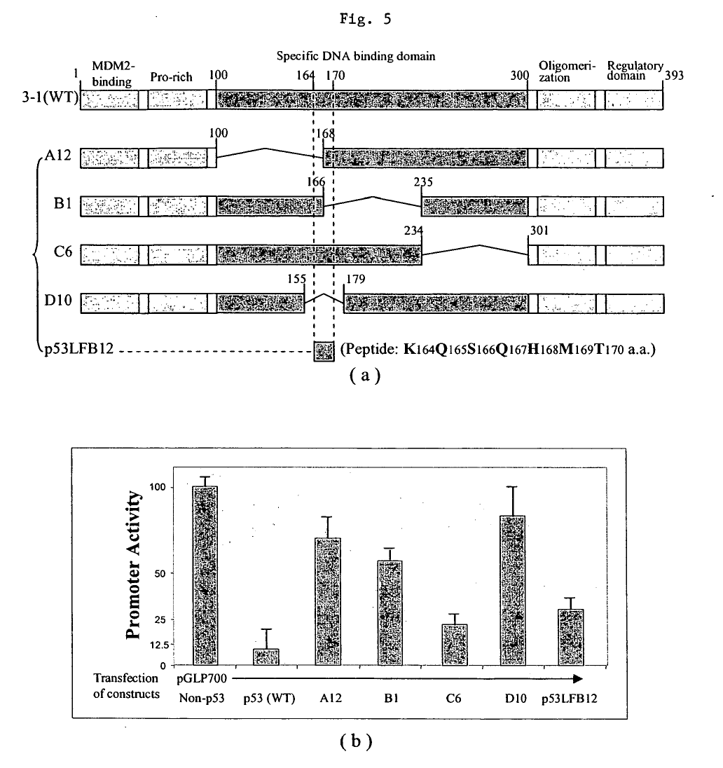

To determine which p53-binding domain inhibits LITAF promoter activity, a series of human p53 constructs were created. Diagrams of several of the major constructs, p53-1, A12, B1, C6, D10 and p53LFB12 are shown in FIG. 5A. The proteins extracted from the p53-null H1299 cells transiently transfected with the p53 constructed noted above plus pGLP700 were assessed for luciferase activity. The promoter activity of pGLP700 alone was assigned the maximal value of 100%. In FIG. 5A different lengths of the p53 DNA were truncated and inserted into the pcDNA3.1/V5-His TOPO vector. The region representing aa 100-300, covering the p53 specific DNA binding sequence, is indicated in each construct by a grey-filled box. Deletions, indicated by ̂, correspond to aa 101-167 in construct A12, aa 167-234 in B1, aa 235-300 in C6 and aa 156-178 in D10. Construct p53LFB12 contained only 7 aa (KQSQHMT [SEQ ID NO: 1]) from the region (aa 164-170).

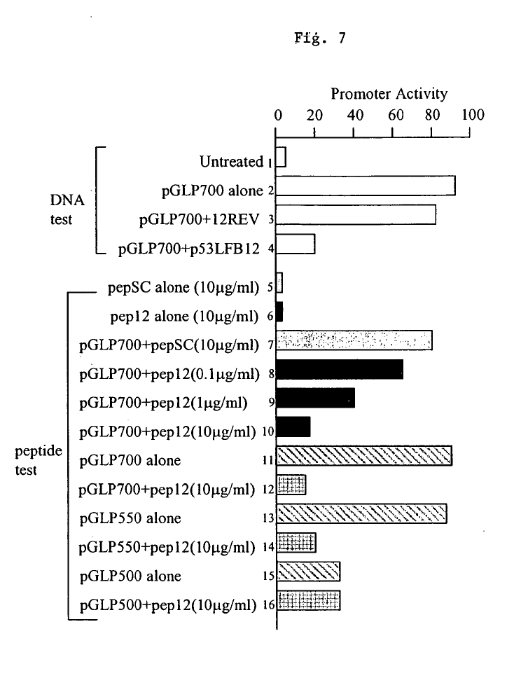

As shown in FIG. 5B, the LITAF promoter activity was strongly reduced to 9% of control due to transient transfection of wild-type p53 (p53-1). The proteins extracted from each transfection of construct plus pGLP700 compared to the transfection of pGLP700 alone in p53-null H1299 cells were measured by luciferase assay. Triplicate assays were performed. The results showed partial LITAF promoter activity was maintained by the expression of wither A 12 (73%) or B1 (61%), which lacked aa 101-167 or aa 167-234, respectively. Construct C6, containing aa 167 and its flanking sequences, reduced LITAF promoter activity to 22% of control even though it lacked aa 235-300. This suggested that the specific residues playing an important role in the inhibition of LITAF promoter activity were located in the region around aa 167. Thus, the DNA clones D10 and p53LFB12, which either possessed (D10) or lacked (p53LFB12) aa 167 and its flanking sequences were constructed. Construct D10, lacking aa 156-178 completely lost its ability to inhibit LITAF promoter activity. In contrast, p53LFB12, which possessed only a short 7 aa peptide representing aa 164 to 170 (KQSQHMT) significantly reduced LITAF promoter activity to 31% of control (FIG. 5B, FIG. 7, condition #4).

### Example 7

### p53 Short Peptide-Mediated Regulation of LITAF Gene Expression

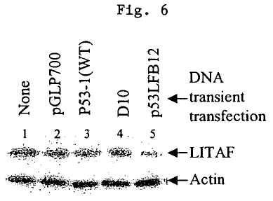

Since the short DNA sequence containing aa KQSQHMT (p53LFB12) in the putative p53 binding domain was found to be essential for the inhibition of LITAF promoter activity by luciferase assay (FIG. 5), it was investigated if whether transfection of this sequence would also affect the regulation of endogenous LITAF gene expression. Thus, the DNAs of p53-1, D10 or p53LFB12, as test samples, and non-DNA or pGLP700 alone as negative controls, were respectively transfected into p53-null H1299 cells. The protein extracts from these transfected cells were analyzed by Western blot with antibodies against LITAF and actin as control. As shown in FIG. 6, the protein level of endogenous LITAF gene expression was not changed by the transient transfection of D10 (lane 4) or of pGLP700 (lane 2) but was greatly reduced by transient transfection of p53-1 (lane 3) or p53LFB12 (lane 5) compared to the untreated control (lane 1). These results show that the level of LITAF protein maintained in p53-null H1299 cells is specifically downregulated by p53 production that contains a short peptide (aa KQSQHMT).

Furthermore, short peptides (pep12) that contain 7 residues (aa 164-170) (KQSQHMT [SEQ ID NO: 1]) corresponding to p53LFB12 and another peptide (pepSC) with reverse sequence of pep12 as a negative control, were synthesized and analyzed using the luciferase assay. Cultures of p53-null H1299 cells (5×10⁶) were transfected with pGLP700 (#2-4, #7-12), pGLP550 (#13 and 14) or pGLP500 (#15 and 16) plus p53LFB12 (#4) or plus 12REV (#3) as control using Lipofactamine 2000 (Invitrogen) for 3 hours, washed with PBS and further transfected with peptide, 10 μg/ml (#10, 12, 14 and 16), then cells were incubated in appropriate medium at 37° C. overnight. Luciferase activities of the lysates from each culture of treated cells were measured. The extracts from untreated (#1), pepSC alone (#5), pep12 alone (#6), pGLP700 alone (#2 and 11, pGLP550 alone (#13) or pGLP500 alone (#15) were used as controls. Triplicate assays were performed.

As shown in FIG. 7, treatment with varying amounts of pep12 (FIG. 7, #8-10, 0.1 μg/ml or 10 μg/ml) plus pGLP700 caused a gradual but significant decrease (66% for #8, 40% for #9 or 15% for #10) in LITAF promoter activity compared to the controls untreated with peptide (100% for #2) or untreated with 10 μg/ml pepSC (95% for #7). Treatment with peptide alone without pGLP700 did not affect the assay (1.5% for #5 or 2.3% for #6). In order to determine which region of the LITAF promoter is the specific site for p53 binding, introduction of pep12 (10 μg/ml) plus pGLP700 (#12) of pGLP550 (#14) significantly reduced LITAF promoter activity to 15% or 23% of control. However, the same treatment with pep12 plus pGLP500 did not affect LITAF promoter activity (#16) compared to the control (#15). These findings suggest that the site from −550 to −500 in the LITAF promoter is specific for pep12 binding activity.

### Example 8

### Analysis of p53 Short Peptide-Mediated LPS-Induced LITAF of Human TNF-α Production in Human Monocytes

To further examine the effects of pep12 on LPS-induced LITAF gene expression and LITAF-dependent TNF-α production, analyses of Western blot and ELISA were performed. Human monocytes (1×10⁶) were stimulated with 0.1 μg/ml E. coli LPS for 3 hours and washed with PBS prior to treatment with peptides for 16 hours with either pep12 (#5) or pepSC as control (#4) using a Chariot kit (Active motif, Carlsbad, Calif.) or transfected with no DNA (#1) or with pGLP700 DNA (#2) as negative controls or with p53-1 DNA (#3) as a positive control. The cells were incubated in appropriate medium at 37° C., 5% CO₂ overnight. The supernatants from cell cultures were measured by ELISA (Abraxis, Warminster, Pa.) and the extracts were detected by Western blot with antibodies against LITAF and actin (as a control).

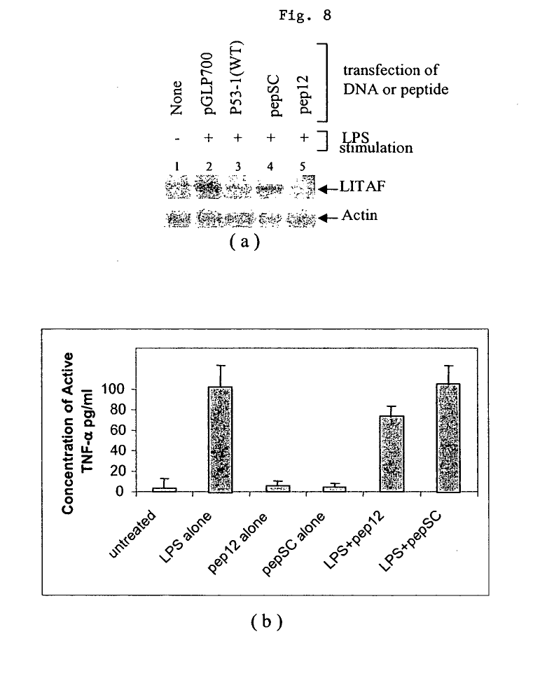

As shown in FIG. 8A, the treatment with pepSC could not affect LPS-induced TNF-α secretion. In contrast, treatment with pep12 significantly reduced TNF-α production to ≦70% of control. Similarly, the results in FIG. 8B from another measurement by Western blot showed the inhibition of LPS-induced LITAF gene expression by pep12 (lane 5) compared to controls (lanes 1, 2 or 4).

### Example 9

### Bioplex® Human Monocyte Assay

This assay was performed to determine the effectiveness of the peptide of the present invention [pep12: SEQ ID NO.: 1] to inhibit various human cytokines. The assay performed was a human monocyte assay well known to those practiced in the art. The kit used here (trade name Bioplex) was supplied by Bio-Rad (Hercules, Calif.). Other commercially available assays are known and suitable for use in the assay described here.

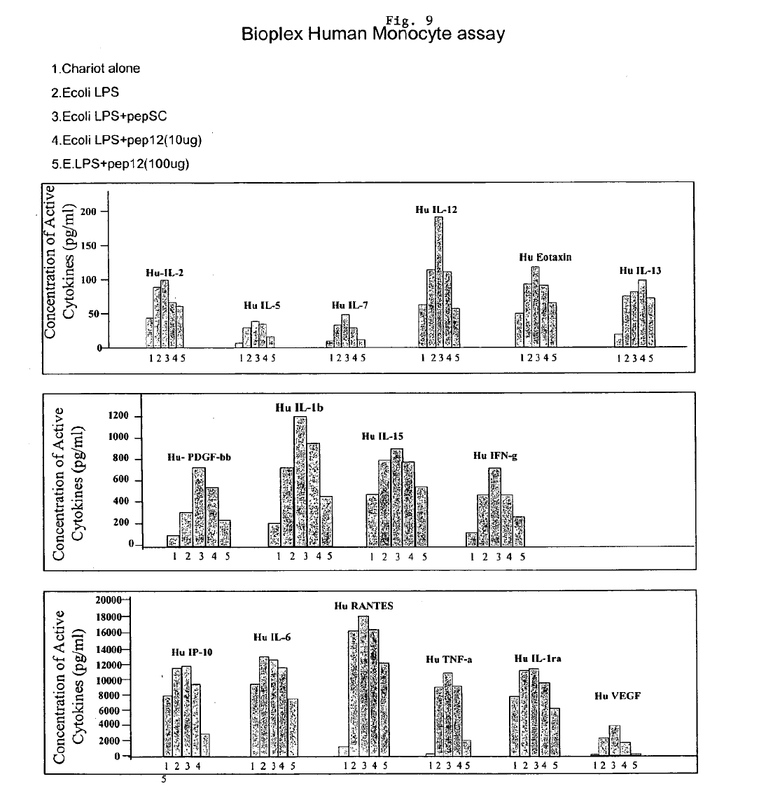

Results are shown in FIGS. 9A, 9B and 9C. Bar (1) is the carrier solution alone (chariot alone); bar (2) is E. coli LPS stimulant; bar (3) is E. coli LPS+pepSC (scramble peptide); bar (4) is E. coli LPS+pep12 (10 υg); bar (5) is E. coli LPS+pep12 (100 υg). The cytokines tested include Human (Hu) IL-2, Hu Il-5, Hu Il-7, Hu Il-12 Hu Eotaxin, Hu Il-13, Hu-PDGF-β, Hu IL-1β, Hu-15, Hu IFN-γ, Hu IP-10, Hu IL-6, Hu RANTES, Hu TNF-α, Hu Il-1ra and Hu VEGF. Most of the cytokines tested showed significant reduction in the concentration of active cytokine in the assay with the exceptions of Hu IL-13, Hu PDGF-β and Hu RANTES. These cytokines showed only minor reductions in active cytokines.

We claim:

1. An isolated and purified peptide comprising the amino acid sequence KQSQHMT [SEQ ID NO: 1].

2. An isolated and purified nucleic acid sequence encoding the amino acid sequence KQSQHMT [SEQ ID NO: 1].

3. An expression vector containing, in expressible form, a nucleic acid sequence encoding a peptide comprising the amino acid sequence KQSQHMT [SEQ ID NO: 1].

4. An antibody that is reactive with a peptide comprising the amino acid sequence KQSQHMT [SEQ ID NO: 1].

5. The amino acid sequence of claim 1, wherein said amino acid sequence comprises an additional 0 to 100 amino acids at the amino-terminal and/or an additional 0 to 100 amino acids at the carboxy-terminal.

6. A method for modulating LITAF (LPS-Induced TNF-alpha Factor) promoter activity in a target cell, said method comprising:
introducing into a cell in need of modulating of LITAF promoter activity a peptide comprising the amino acid sequence KQSQHMT [SEQ ID NO: 1].

7. The method of claim 6, wherein said amino acid sequence KQSQHMT [SEQ ID NO: 1] is encoded by an expression vector suitable for the expression of the amino acid sequence KQSQHMT [SEQ ID NO: 1] in the target cell.

8. The method of claim 6, wherein said amino acid sequence comprises an additional 0 to 100 amino acids at the amino-terminal and/or an additional 0 to 100 amino acids at the carboxy-terminal.

9. A method for modulating TNF-alpha in a target cell, said method comprising:
introducing into a cell in need of modulating of TNF-alpha production a peptide comprising the amino acid sequence KQSQHMT [SEQ ID NO: 1].

10. The method of claim 9, wherein said amino acid sequence KQSQHMT [SEQ ID NO: 1] is encoded by an expression vector suitable for the expression of the amino acid sequence KQSQHMT in the target cell.

11. The method of claim 9, wherein said amino acid sequence comprises an additional 0 to 100 amino acids at the amino-terminal and/or an additional 0 to 100 amino acids at the carboxy-terminal.

12. A method for modulating IL-1-beta production in a target cell, said method comprising:
introducing into a cell in need of modulating of IL-1-beta production a peptide comprising the amino acid sequence KQSQHMT [SEQ ID NO: 1].

13. The method of claim 12, wherein said amino acid sequence KQSQHMT [SEQ ID NO: 1] is encoded by an expression vector suitable for the expression of the amino acid sequence KQSQHMT [SEQ ID NO: 1] in the target cell.

14. The method of claim 12, wherein said amino acid sequence comprises an additional 0 to 100 amino acids at the amino-terminal and/or an additional 0 to 100 amino acids at the carboxy-terminal.

15. A method for increasing VEGF production in a target cell, said method comprising: introducing into a cell in need of an increase of VEGF production a peptide comprising the amino acid sequence KQSQHMT [SEQ ID NO: 1].

16. The method of claim 15, wherein said amino acid sequence KQSQHMT [SEQ ID NO: 1] is encoded by an expression vector suitable for the expression of the amino acid sequence KQSQHMT [SEQ ID NO: 1] in the target cell.

17. The method of claim 15, wherein said amino acid sequence comprises an additional 0 to 100 amino acids at the amino-terminal and/or an additional 0 to 100 amino acids at the carboxy-terminal.

18. A method for modulating the production of one or more cytokines in a cell, said cytokines selected from a group consisting of, TNF-alpha, IL-1-eta, VEGF, IL-2, IL-5, IL-7, IL-12, Eotaxin, IL-15, IFN-γ, IP-10 (human interferon-inducible protein-10), IL-6, RANTES and IL-1ra.in a target cell, said method comprising:
introducing into a cell in need of modulation of pro-inflammatory cytokine production a peptide comprising the amino acid sequence KQSQHMT [SEQ ID NO: 1].

19. The method of claim 9, wherein said amino acid sequence KQSQHMT [SEQ ID NO: 1] is encoded by an expression vector suitable for the expression of the amino acid sequence KQSQHMT in the target cell.

20. The method of claim 9, wherein said amino acid sequence comprises an additional 0 to 100 amino acids at the amino-terminal and/or an additional 0 to 100 amino acids at the carboxy-terminal.

21. A method for identifying a small molecule characterized by the ability to inhibit p53/LITAF binding, said method comprising.
a. providing a library of small molecules to be screened for the ability to inhibit p53/LITAF binding;
b. forming a reaction mixture comprising a small molecule to be screened for the ability to inhibit p53/LITAF binding, and a mixture of the peptide encoded by SEQ ID NO: 1 and the LITAF promoter region or an active fragment thereof;
c. incubating the reaction mixture of step b) for a period of time and under conditions appropriate for p53/LITAF binding;
d. determining the extent of p53/LITAF binding following the incubation of step c);
e. comparing the amount p53/LITAF binding determined in step d) to the amount of p53/LITAF binding detected in an otherwise identical incubation mixture which does not include a small molecule to be screened for the ability to inhibit p53/LITAF binding, a decrease in the binding determined in step d) to that of the otherwise identical incubation mixture being indicative of the small molecule of step b) being characterized by the ability to inhibit p53/LITAF promoter region binding.

22. The method of claim 18 wherein said p53-LITAF binding complex is detected by electrophoresis.

23. The method according to claim 18 wherein said p53-LITAF binding complex is detected by electromobility shift assay (EMSA).

24. A method of promoting angiogenesis comprising introducing the peptide of SEQ ID NO: 1 to a tissue capable of undergoing angiogenesis.

25. The method of claim 21, wherein said SEQ ID NO: 1 is introduced by transfection of an expression construct comprising a nucleotide sequence encoding SEQ ID NO: 1 into one or more cells of said tissue.

26. The method of claim 21, wherein said SEQ ID NO: 1 is administered by the transfection of said protein into said tissue.

27. The method of claim 26, wherein said tissue is located in an organism and said introduction of said SEQ ID NO: 1 is either local or systemic.


SEQUENCE LISTING (58 sequences)

SEQ ID NO: 1 - protein, 7 aa, Artificial Sequence
  Description of Artificial Sequence Synthetic peptide
KQSQHMT

SEQ ID NO: 2 - DNA, 21 bp, Artificial Sequence
  Description of Artificial Sequence Synthetic primer
atggaggagccgcagtcagat

SEQ ID NO: 3 - DNA, 30 bp, Artificial Sequence
  Description of Artificial Sequence Synthetic primer
ccgtcatgtgctgggaagggacagaagatg

SEQ ID NO: 4 - DNA, 19 bp, Artificial Sequence
  Description of Artificial Sequence Synthetic primer
tcagtctgagtcaggccct

SEQ ID NO: 5 - DNA, 21 bp, Artificial Sequence
  Description of Artificial Sequence Synthetic primer
atggaggagccgcagtcagat

SEQ ID NO: 6 - DNA, 19 bp, Artificial Sequence
  Description of Artificial Sequence Synthetic primer
tcagtctgagtcaggccct

SEQ ID NO: 7 - DNA, 21 bp, Artificial Sequence
  Description of Artificial Sequence Synthetic primer
atggaggagccgcagtcagat

SEQ ID NO: 8 - DNA, 30 bp, Artificial Sequence
  Description of Artificial Sequence Synthetic primer
acatgtagttctgtgactgcttgtagatgg

SEQ ID NO: 9 - DNA, 21 bp, Artificial Sequence
  Description of Artificial Sequence Synthetic primer
aactacatgtgtaacagttcc

SEQ ID NO: 10 - DNA, 19 bp, Artificial Sequence
  Description of Artificial Sequence Synthetic primer
tcagtctgagtcaggccct

SEQ ID NO: 11 - DNA, 21 bp, Artificial Sequence
  Description of Artificial Sequence Synthetic primer
atggaggagccgcagtcagat

SEQ ID NO: 12 - DNA, 19 bp, Artificial Sequence
  Description of Artificial Sequence Synthetic primer
tcagtctgagtcaggccct

SEQ ID NO: 13 - DNA, 21 bp, Artificial Sequence
  Description of Artificial Sequence Synthetic primer
atggaggagccgcagtcagat

SEQ ID NO: 14 - DNA, 30 bp, Artificial Sequence
  Description of Artificial Sequence Synthetic primer
tgctccctgggtagtggatggtggtacagt

SEQ ID NO: 15 - DNA, 21 bp, Artificial Sequence
  Description of Artificial Sequence Synthetic primer
ccagggagcactaagcgagca

SEQ ID NO: 16 - DNA, 19 bp, Artificial Sequence
  Description of Artificial Sequence Synthetic primer
tcagtctgagtcaggccct

SEQ ID NO: 17 - DNA, 21 bp, Artificial Sequence
  Description of Artificial Sequence Synthetic primer
atggaggagccgcagtcagat

SEQ ID NO: 18 - DNA, 19 bp, Artificial Sequence
  Description of Artificial Sequence Synthetic primer
tcagtctgagtcaggccct

SEQ ID NO: 19 - DNA, 21 bp, Artificial Sequence
  Description of Artificial Sequence Synthetic primer
atggaggagccgcagtcagat

SEQ ID NO: 20 - DNA, 21 bp, Artificial Sequence
  Description of Artificial Sequence Synthetic primer
ggtgccgggcgggggtgtgga

SEQ ID NO: 21 - DNA, 15 bp, Artificial Sequence
  Description of Artificial Sequence Synthetic primer
catgagcgctgctca

SEQ ID NO: 22 - DNA, 19 bp, Artificial Sequence
  Description of Artificial Sequence Synthetic primer
tcagtctgagtcaggccct

SEQ ID NO: 23 - DNA, 21 bp, Artificial Sequence
  Description of Artificial Sequence Synthetic primer
atggaggagccgcagtcagat

SEQ ID NO: 24 - DNA, 19 bp, Artificial Sequence
  Description of Artificial Sequence Synthetic primer
tcagtctgagtcaggccct

SEQ ID NO: 25 - DNA, 25 bp, Artificial Sequence
  Description of Artificial Sequence Synthetic primer
atgaagcagtcacagcacatgacga

SEQ ID NO: 26 - DNA, 25 bp, Artificial Sequence
  Description of Artificial Sequence Synthetic primer
cgtcatgtgctgtgactgcttcata

SEQ ID NO: 27 - DNA, 25 bp, Artificial Sequence
  Description of Artificial Sequence Synthetic primer
atggcagtacacgacactgacgaaa

SEQ ID NO: 28 - DNA, 25 bp, Artificial Sequence
  Description of Artificial Sequence Synthetic primer
ttcgtcagtgtcgtgtactgccata

SEQ ID NO: 29 - DNA, 22 bp, Artificial Sequence
  Description of Artificial Sequence Synthetic primer
ccagagggccgggagcgcccca

SEQ ID NO: 30 - DNA, 22 bp, Artificial Sequence
  Description of Artificial Sequence Synthetic primer
tttacccagcaccggcggtgga

SEQ ID NO: 31 - DNA, 22 bp, Artificial Sequence
  Description of Artificial Sequence Synthetic primer
tttacccagcaccggcggtgga

SEQ ID NO: 32 - DNA, 21 bp, Artificial Sequence
  Description of Artificial Sequence Synthetic primer
gcccccgcccccgtccccgcc

SEQ ID NO: 33 - DNA, 22 bp, Artificial Sequence
  Description of Artificial Sequence Synthetic primer
tttacccagcaccggcggtgga

SEQ ID NO: 34 - DNA, 21 bp, Artificial Sequence
  Description of Artificial Sequence Synthetic primer
ggccagctcagacctcccggc

SEQ ID NO: 35 - DNA, 22 bp, Artificial Sequence
  Description of Artificial Sequence Synthetic primer
tttacccagcaccggcggtgga

SEQ ID NO: 36 - DNA, 21 bp, Artificial Sequence
  Description of Artificial Sequence Synthetic primer
cggcgcggggacgccggggcg

SEQ ID NO: 37 - DNA, 22 bp, Artificial Sequence
  Description of Artificial Sequence Synthetic primer
tttacccagcaccggcggtgga

SEQ ID NO: 38 - DNA, 21 bp, Artificial Sequence
  Description of Artificial Sequence Synthetic primer
ggtggcgccagcacctgctgg

SEQ ID NO: 39 - DNA, 22 bp, Artificial Sequence
  Description of Artificial Sequence Synthetic primer
tttacccagcaccggcggtgga

SEQ ID NO: 40 - DNA, 21 bp, Artificial Sequence
  Description of Artificial Sequence Synthetic primer
gcctcctgggatgccaggggg

SEQ ID NO: 41 - DNA, 22 bp, Artificial Sequence
  Description of Artificial Sequence Synthetic primer
tttacccagcaccggcggtgga

SEQ ID NO: 42 - DNA, 21 bp, Artificial Sequence
  Description of Artificial Sequence Synthetic primer
gcccccgcccccgtccccgcc

SEQ ID NO: 43 - DNA, 21 bp, Artificial Sequence
  Description of Artificial Sequence Synthetic primer
ccccagccaagggctcagtgc

SEQ ID NO: 44 - DNA, 21 bp, Artificial Sequence
  Description of Artificial Sequence Synthetic primer
gcccccgcccccgtccccgcc

SEQ ID NO: 45 - DNA, 21 bp, Artificial Sequence
  Description of Artificial Sequence Synthetic primer
ctggcgccaccggccccccgc

SEQ ID NO: 46 - DNA, 22 bp, Artificial Sequence
  Description of Artificial Sequence Synthetic primer
gcgcccgagaggccagctcaga

SEQ ID NO: 47 - DNA, 21 bp, Artificial Sequence
  Description of Artificial Sequence Synthetic primer
ccccagccaagggctcagtgc

SEQ ID NO: 48 - DNA, 100 bp, Artificial Sequence
  Description of Artificial Sequence Synthetic primer
gcccccgcccccgtccccgccgcccggcccttttctcggggcgcccgagaggccagctcagacctcccggctcgacaggcggcgcgggcggcggtgagtg

SEQ ID NO: 49 - DNA, 78 bp, Artificial Sequence
  Description of Artificial Sequence Synthetic primer
ccccagccaagggctcagtgcccggggcccccagcaggtccccggcgtccccgcgccgcactcaccgccgcccgcgcc

SEQ ID NO: 50 - DNA, 90 bp, Artificial Sequence
  Description of Artificial Sequence Synthetic primer
gcccccgcccccgtccccgccgcccggcccttttctcggggcgcccgagaggccagctcagacctcccggctcgacaggccggggaccag

SEQ ID NO: 51 - DNA, 90 bp, Artificial Sequence
  Description of Artificial Sequence Synthetic primer
ccccagccaagggctcagtgcccggggcccccagcaggtgctggcgccaccggccccccgctgtctcccgctggtccccggcctgtcgag

SEQ ID NO: 52 - DNA, 90 bp, Artificial Sequence
  Description of Artificial Sequence Synthetic primer
gcccccgcccccgtccccgccgcccggcccttttctcggggcgcgggcggcggtgagtgcggcgcggggacgccggggcgcggggaccag

SEQ ID NO: 53 - DNA, 89 bp, Artificial Sequence
  Description of Artificial Sequence Synthetic primer
cccagccaagggctcagtgcccggggcccccagcaggtgctggcgccaccggccccccgctgtctcccgctggtccccgcgccccggcg

SEQ ID NO: 54 - protein, 7 aa, Artificial Sequence
  Description of Artificial Sequence Synthetic peptide
SKMQQTH

SEQ ID NO: 55 - DNA, 26 bp, Artificial Sequence
  Description of Artificial Sequence Synthetic oligonucleotide
aaacaaaaccaaaacaaacaaaaaaa

SEQ ID NO: 56 - DNA, 1242 bp, Homo sapiens
ccagagggccgggagcgccccagcgcgggtccggggtcgaggttggtggtcccgccgcctggaagtggtggagcggcgcggggaggggggaaagtggggagggccgcgagcggcaaatcaggagggagggccaggctgtcgggggcggacgggtggggctggcaccgagaggcaggggcaggataggagagtcggggagacaccaggtccgcgccaggcgtctcgggggtaagcctgggaagctctggggtggcgagcgagccgggtgtgggatggagcctgggggcgatcagagtgaggcggggaaccggcgatggtctcgcctggggcccaggtgccggccgcgggggcgggagaagccggaagcccaagcccgccgcgctccgccccggcgggtccagcccccgcccccgtccccgccgcccggcccttttctcggggcgcccgagaggccagctcagacctcccggctcgacaggcggcgcgggcggcggtgagtgcggcgcggggacgccggggcgcggggaccagcgggagacagcggggggccggtggcgccagcacctgctgggggccccgggcactgagcccttggctggggcctcctgggatgccagggggcgcgggtcgggtcgcgggcatcgaggcgcggcggagggcgtgggggcccggccggggcggggtccggcctcccagcgctggtcccggccgcgtctccggttgggttcagctcctgcgtcccagagtggcccgatcgcgcgtggcggggtcgtccggcccccacccgaacgagcgcccttcgcggcccgccgcgtccccctccccggagaggacggcccctgggctttttagaaaaaggcgcgattctctctagtgactcaggttgagatttccagaaatatcccccgggggttcagaaacaaaaccaaaacaaacaaaaaaaccccaacgaattcccaaatgctatttgccaaacatttgacttcttaggggcgcgggtacccgcgtttctctccctgcccccgcgacttcgcgcaagatccgggaaggacacccgaggcccctgggagaccctggggaggtgaaaatcagagagcgaagcgggccgtggcccctaggcctgacccctccccgcggggtaaggcgggcaccccgcgagcgcaggggtcctcttactgctgatggcacccagctctgggcccagacgccgctcaccgtccaccgccggtgctgggtaaaatg

SEQ ID NO: 57 - DNA, 20 bp, Artificial Sequence
  Description of Artificial Sequence Synthetic primer
cacatgacggaggttgtgag

SEQ ID NO: 58 - DNA, 21 bp, Artificial Sequence
  Description of Artificial Sequence Synthetic primer
cggggaaccggcgatggtctc